(19) United States
(12) Patent Application Publication
(10) Pub. No.: US 2025/0241850 A1
(43) Pub. Date: Jul. 31, 2025

(54) BIOADHESIVE NASAL SPRAY COMPOSITION, PREPARATION METHOD AND USE

(71) Applicant: Eurofarma Laboratórios S.A., Itapevi, São Paulo (BR)

(72) Inventors: George SERRATO GUALBERTO, São Paulo (BR); Marcos MARIANO, São Roque (BR)

(73) Assignee: Eurofarma Laboratórios S.A., Itapevi, São Paulo (BR)

(21) Appl. No.: 18/855,004

(22) PCT Filed: Mar. 15, 2023
(86) PCT No.: PCT/BR2023/050090

§ 371 Date: Oct. 8, 2024

(30) Foreign Application Priority Data
Apr. 8, 2022 (BR) 1020220068550

(51) Int. Cl. A61K 9/00; A61K 31/439; A61K 47/12; A61K 47/18; A61K 47/26; A61K 47/38

(52) U.S. Cl. CPC A61K 9/0043; A61K 31/439; A61K 47/12; A61K 47/186; A61K 47/26; A61K 47/38

(57) ABSTRACT

The present invention relates to a bio-adhesive nasal spray composition comprising granisetron hydrochloride, resulting in a bio-adhesive system for systemic delivery, as well as the process for obtaining it and the use thereof for the manufacture a medicament in the form of a bio-adhesive nasal spray.

### FIELD OF THE INVENTION

The present invention relates to a preparation in the form of a nasal spray suspension comprising granisetron hydrochloride and micro/nanofibrillated cellulose in a bio-adhesive system for systemic delivery, as well as its process for obtaining and use thereof. The present invention is in the fields of Pharmaceutical Technology and Pharmacotechnics.

### BACKGROUND OF THE INVENTION

Chemotherapy-induced nausea and vomiting are the most serious side effects of anticancer drugs. Nausea is a feeling of discomfort and the urge to vomit. Vomiting is the forceful expulsion of stomach contents through the mouth. Antiemetic agents are drugs that are intended to treat nausea and vomiting and are usually given orally or intravenously (IV).

Granisetron is a serotonin 5-HT3 receptor antagonist used as an antiemetic in the treatment of nausea and vomiting after chemotherapy and radiotherapy. Its main effect is to reduce the activity of the vagus nerve, the nerve that activates the vomiting center in the medulla oblongata, without having a major effect on nausea, since it does not act on dopamine receptors or muscarinic receptors.

Granisetron was developed by the British pharmaceutical company Beecham, around 1988. The drug was approved in 1998, in the United States, by the local health agency-Food and Drug Administration (FDA)-under the name Kytril® and marketed by Hoffmann La Roche.

Granisetron hydrochloride (GRA HCl) has a short half-life (3-4 h). Therefore, its administration by both the oral and intravenous (IV) routes presents disadvantages due to the need for frequent administration of the drug. Particularly in any situation where a patient is suffering from nausea and vomiting, oral administration of an antiemetic agent is challenging and creates increased discomfort due to difficulty in swallowing and/or the ability to maintain the drug in the gastrointestinal system long enough for adequate absorption. For patients unable to swallow tablets due to emesis, intravenous antiemetics are mandatory. However, for the intravenous or intramuscular route, home administration is usually prohibitive.

In this sense, products with nasal administration for systemic treatment are an option because they are easy, painless, and comfortable to administer for the patient, which results in better adherence to treatment.

Intranasal (IN) drug administration has attracted increasing interest due to its potential to avoid first-pass metabolism, a drug action step in which a large part of the administered dose is removed from the systemic circulation by the liver, and to favor the arrival of the drug in the central nervous system (CNS) without the need to cross the blood-brain barrier (BBB). In addition, the large surface area of the nasal cavities, approximately 150 cm², helps the drug to be absorbed in therapeutically effective amounts. The adoption of other non-invasive systemic administration methods requires that the drug enter the CNS exclusively through the BBB, whereas intranasal administration has three possible permeation routes: the systemic route itself, the olfactory nerve, and the trigeminal nerve.

Systemic administration of drugs via the intranasal route allows them to reach the brain by diffusion in the blood through the vesicular network of the nasal canal and then pass the BBB. However, hydrophilic drugs and high molecular weight molecules are inefficiently transported by this route due to the highly selective and hydrophobic nature of the BBB. Hydrophilic drugs tend to be easily dissolved and solubilized, thus allowing their absorption. Thus, hydrophilic drugs with adequate permeability are more likely to be absorbed in the gastrointestinal tract and exert their therapeutic effects. To reach the brain, hydrophilic drugs can be absorbed by the olfactory (olfactory pathway) and trigeminal (trigeminal pathway) nerves. In the olfactory pathway, drugs are transported along the axon, using the paracellular or transcellular pathway, to the olfactory cortex and then to the cerebrum and cerebellum. Through the trigeminal pathway, drugs diffuse into the maxillary and ophthalmic branches of the nerve and enter the brain stem.

Despite these advantages, the IN route may reduce drug bioavailability due to mucociliary clearance and enzymatic degradation in the nasal cavity. Nasal absorption is directly dependent on the drug's residence time in the mucosa. Lower absorption leads to lower bioavailability.

Mucociliary clearance removes bacteria, viruses, allergens, and dust from the respiratory tract, making it an important cleansing mechanism and “first line of defense” against respiratory tract infections. This same clearance, however, limits the residence time of a compound in the nasal cavity to about 15 minutes.

Thus, one of the challenges of intranasal drug administration, especially hydrophilic drugs, is the low bioavailability resulting from low membrane permeability and rapid elimination by mucus. The mucosa/epidermis constitutes a barrier to the permeation of hydrophilic and charged substances, especially negatively charged ones.

The choice of the IN route is only appropriate for drugs in low doses and for those that are soluble in aqueous medium and capable of passing through the mucous layer in a timely manner so as not to be eliminated together with the nasal mucus. Additionally, products for nasal use should not cause local irritation or interfere with the normal physiology of the region.

In addition to the factors mentioned, the absorption of drugs administered intranasally is affected by a series of specific characteristics of the composition and of the drug itself, such as weight and/or molecular structure, solubility, lipophilicity, ionization, pH, osmolarity and viscosity. When the molecular weight is below 300 Daltons (Da), most drugs can permeate through membranes. Between 300 and 1,000 Da, absorption is influenced by the molecular structure and, when it exceeds 1,000 Da, absorption decreases rapidly. Granisetron hydrochloride is at the lower limit of this second range, with approximately 348 g·mol⁻¹.

Nasal aerosols (sprays) are the most common intranasal administration system for the delivery of drugs with local and systemic action. They are generally compositions as a solution and/or suspension, packaged in specific devices, which allow control of the dose volume, contained in a dosing chamber inside the spray and atomization valve via compression of the actuator, obtaining droplets ranging from 50-140 μL per spray after initial activation.

Nasal spray compositions may contain buffers or suspending agents, mineral acids and bases, and preservatives, and the most commonly used vehicle is an aqueous solution. This is perhaps the simplest and most convenient form of composition and is practical in different types of administration devices (sprays and drops). Environmental conditions (such as temperature, light, etc.) are determining factors in the stability of the product, and in this sense, a powder composition could be more suitable, due to greater physical stability and the possibility of not containing preservatives. However, it could cause nasal irritation and a sensation of sand in the nose.

In view of the disadvantages of aqueous and powder formulations, formulations presented as nasal gel, solutions or thickened suspensions of high viscosity are forms of delivery that have been explored in the intranasal administration of drugs, since they have many advantages, such as reduced postnasal drip and anterior nostril leakage after application, and low irritation of the nasal mucosa. Despite this, gels tend to present difficulty in delivering an exact dose of the active substance.

Bio-adhesive and thickening materials are used to extend the retention time of the formula on the mucosa, allowing better drug absorption. In general, retention on the mucosa is correlated with greater viscosity, however, increased viscosity can have a negative impact on the quality of the spray generated, since it will become increasingly difficult to generate droplets of an adequate size, in addition to not interrupting the fluid flow causing the runoff.

Conventional excipients for pH control and improvement of organoleptic characteristics, such as synthetic sweeteners, osmotic correction agents and preservatives, are widely discussed. However, many of the systems described fail to offer robust rheological characteristics, causing several problems, such as heterogeneity of drug dosage, phase separation of the final product and low capacity to be dispersed as droplets during the “atomization” caused by the use of a spray.

Technologically, the challenges of intranasal administration and subsequent delivery to the brain are many and include the reliable delivery of the atomized product to the olfactory nerve endings located at the apex of the nasal cavity. One possible approach is the development of a device capable of generating a narrow spray cloud directed towards the olfactory region when inserted into the nostrils. There are, however, several physicochemical and rheological characteristics of this vehicle that can influence and interfere with the efficiency of the product via the intranasal route, as indicated below:
Improving absorption: To improve absorption, several changes in the composition properties can be altered. Changing the dosage form, such as changing to a powder or gel, by using bio-adhesives or absorption-enhancing agents or changing the viscosity could increase systemic uptake. Strategies such as mucous adhesion have gained interest due to their ability to prolong the mucosal residence time of drug delivery systems. Mucous adhesion is defined as the state in which a material and mucus or a mucous membrane are held together for an extended period of time by interfacial forces. Mucous adhesive drug delivery systems can induce high local concentration of the drug by maintaining intimate contact at the absorption site;
Isotonicity: An isotonic solution is preferably the best nasal solution, because hypertonicity will lead to shrinkage of the nasal mucosa;
Suspension rheology: Physical characteristics of the active substance vehicle, such as viscosity, have controversial effects. Initially, a higher viscosity increases the contact time with the nasal mucosa (increasing the permeation time), probably resulting in better absorption. However, in some cases, a highly viscous composition may delay the permeation of the drug molecule through the mucous layer on top of the nasal epithelial cells, disrupting nasal absorption. Furthermore, a viscous composition may disrupt mucociliary clearance. Consequently, optimizing the rheological behavior of the vehicle is essential;
Nasal delivery and absorption: Systemic uptake may be enhanced by a longer residence time and a wider spread of the drug over the mucosa. With respect to a longer residence time, the elimination of a spray is much slower than that of drops since most of the spray is deposited in the non-ciliated areas. Although the distribution and elimination of drops are less predictable than after spraying, a shorter residence time is seen, mainly because the droplet solution spreads more widely over the ciliated area. That is, a larger distributed area will improve systemic absorption, as observed in tests with deposition of nasal products in both nostrils compared to one nostril. The best deposition site in the nose is debatable and depends on the drug properties;
Volume: the spread of the product appears to improve the nasal absorption of a drug with low intranasal absorption, since two doses of each 50 μL appear to be more efficient than a single dose of 100 μL;
Device: again, when comparing systemic uptake after drop or spray administration, better uptake after spray administration was observed in several studies; and
Route of absorption: Physiological and histological studies in animals and humans have shown that the mucosa in the upper part of the nose is connected with the cerebral perivascular spaces and the subarachnoid spaces of the olfactory lobes of the brain, which would make this route of drug transport viable.

It is clear, therefore, that there are many challenges to be overcome in the development of an intranasal spray composition that acts on the central nervous system. Such challenges reflect the lack of a drug that mitigates the adverse effects of anti-cancer therapy, specifically nausea and vomiting, which is easy to administer and has a rapid onset of action, while at the same time not requiring swallowing, which is often difficult in patients with such symptoms.

In this way, the prior art benefits from the teachings proposed herein which, given the difficulties of the technical field in question, present novelty and inventive step.

### BRIEF DESCRIPTION OF THE FIGURES

FIG. 1, taken from the prior art documents (Pinkey, S.; Skuse, D.; Rowson, N.; and Blackburn, S., in “Microfibrillated cellulose-a new structural material”, no publication date available) shows in (a) and (b), the description of the structure of the cellulose fiber and, in (c) and (d) the structure obtained after processing for fibrillation. In (c), the processing for fibrillation was carried out by the acid hydrolysis+sonication method (method 1), while in (d), the mechanical shearing method (method 2) was used, which produced microcrystalline fibrils with a specific size in the range of 100 nm-300 nm, or enzymatic hydrolysis+mechanical shearing (method 3), which produced nanofibrillated cellulose with a size of several micrometers.

FIG. 2 shows in (a) optical microscopy images; and in (b) and (c) atomic force microscopy images representing the different micro and nanometric structures of the system.

FIG. 3 shows the flowchart of the manufacturing process of the bio-adhesive nasal spray composition of the present invention but is not limited to this.

FIG. 4 illustrates the assessment of adhesion and flow of the composition presented in Table 1 at different temperatures.

FIG. 5 shows a viscosity curve corresponding to a micro/nanocellulose suspension obtained through the process of the present invention.

FIG. 6 shows the correlation between different concentrations of microcrystalline/fibrillated cellulose dispersion with viscosity and flow time from a method using a capillary viscometer.

FIG. 7 shows the values (a) and viscometry graph (b) related to the viscosity obtained in relation to shear using different grades of microcrystalline cellulose in suspension at 10% w/v.

FIG. 8 shows the plume pattern and geometry obtained using Sprayview equipment (Proveris Scientific, USA). Scan images were obtained at different capture distances to gather product characterization data.

### SUMMARY OF THE INVENTION

The present invention aims to present an optimized composition of a product as a nasal spray to be used in the administration of the active ingredient granisetron hydrochloride. Specifically, the present invention aims to present a composition in a nasal spray gel with a viscosity suitable for absorption by the nasal mucosa, which prevents runoff, and which allows for adequate dispersion of the droplets formed by atomization so that there is wide coverage of the nasal cavity and mucosa, while allowing for rapid elastic recovery by drastically increasing the viscosity at rest.

The present invention shows the following objects as inventive concepts.

The present invention has as its first object, a pharmaceutical composition in bio-adhesive nasal spray, comprising:
(a) at least one active pharmaceutical ingredient;
(b) at least one tonicity agent;
(c) at least one preservative;
(d) at least one buffering system;
(e) at least one rheological modifier;
(f) at least one pseudoplastic thickening polymer; and
(g) micro/nanofibrillated cellulose.

The present invention has as a second object the process of preparing a nasal spray composition, as defined in the first object and in its embodiments, comprising the steps of:
(a) Preparation of the buffer system containing a thickening polymer;
(b) Addition of the active ingredient to the aqueous buffer system;
(c) Inclusion of a thermogelling agent and a preservative agent in the active ingredient;
(d) Preparation of micro/nanofibrillated cellulose;
(e) Mixing and homogenization of the systems described above; and
(f) Adjustment of final volume via addition of purified water.

In a third object, the present invention shows the use of the bio-adhesive nasal spray composition, as defined in the first object and in its embodiments, for the manufacture of a medicament for the treatment of nausea and vomiting.

The differential of the present invention comprises the use of microcrystalline cellulose and internal processing to obtain a mixed system, containing a fraction of fibrillated cellulose in order to have a unique pseudoplastic behavior favorable in nasal formulations.

### Definitions

In the present invention, the expression “nasal spray” refers to a mass of very small liquid droplets forced into the nasal cavities using a special device in order to deliver a certain medicament into the nasal cavity.

In the present invention, the term “micro/nanofibrillated cellulose” refers to a cellulosic mixture containing fractions of particles of micrometric and nanometric dimensions obtained mechanically through intense shearing and temperature increase of an initial suspension of microcrystalline cellulose.

Suspensions are liquid preparations consisting of solid particles dispersed in a liquid phase in which such particles are not soluble. In the present invention, the term “suspension” refers to a dispersion of cellulosic particles, on the micro and nanometric scales, in an aqueous medium.

### DETAILED DESCRIPTION OF THE INVENTION

The present invention relates to an optimized composition of a product as a nasal spray developed from the rheological properties of a suspension containing micro/nanocellulose to be used in the administration of the active ingredient granisetron hydrochloride.

The present invention has as its first object a pharmaceutical composition in bio-adhesive nasal spray, comprising:
(a) at least one active pharmaceutical ingredient;
(b) at least one tonicity agent;
(c) at least one preservative;
(d) at least one buffering system;
(e) at least one rheological modifier;
(f) at least one pseudoplastic thickening polymer; and
(g) micro/nanofibrillated cellulose.

In an embodiment, the bio-adhesive nasal spray pharmaceutical composition comprises:
(a) at least one active pharmaceutical ingredient in a range of 0.01-5 wt % of the composition;
(b) at least one tonicity agent in a range of 0.1-20 wt % of the composition;
(c) at least one preservative in a range of 0.001-5 wt % of the composition;
(d) at least one buffering system in a range of 0.1-5 wt % of the composition;
(e) at least one rheological modifier in a range of 0.01-10 wt % of the composition;
(f) at least one pseudoplastic thickening polymer in a range of 0.01-25 wt % of the composition; and
(g) micro/nanofibrillated cellulose in a range of 3.0-4.0 by mass of the composition,
final viscosity of which varies from 10⁴-10⁶ cP.

In an embodiment, the tonicity agent (b) is selected from the group consisting of sodium chloride, potassium chloride, glycerin, mannitol, dextrose, isodextrose in association with the drug(s) so as to achieve isotonic and/or slightly hypertonic values.

Isotonicity is a property of the product. It refers to any material in the formula that contributes to increasing the ionic strength of the system. Organic materials, such as sugars and cellulose, have a lower contribution. Ionic materials, such as sodium chloride and the drug itself, have a greater effect. The effect is directly related to the concentration of the components.

In an embodiment, the preservative (c) is selected from the group consisting of cetrimide, cetylpyridinium chloride, chlorhexidine, chlorobutanol, chlorocresol, chloroxylenol, cresol, dimethyl ether, ethylparaben, glycerin, hexetidine, imidazolidinyl urea, methylparaben, phenoxyethanol, phenethyl alcohol, potassium benzoate, potassium metabisulfite, potassium sorbate, propionic acid, propylparaben, sodium benzoate, sodium metabisulfite, and/or thimerosal.

In an embodiment, the buffering system (d) is selected from the group consisting of citric acid, sodium citrate, sodium phosphate monohydrate, sodium phosphate dihydrate, phosphoric acid, hydrochloric acid, sodium hydroxide, ammonium hydroxide, boric acid, and/or sodium borate.

In an embodiment, the rheological modifier (e) is selected from the group consisting of acacia, albumins, sodium carboxymethyl cellulose, carrageenans, microcrystalline cellulose, cellulose acetate, chitosan, dextrins, gelatin, guar gum, hyaluronic acid, hydroxyethyl cellulose, hydroxypropyl starch, hydroxypropyl cellulose, hydroxymethylpropyl cellulose, methyl cellulose, polyethylene glycols, poly(methyl vinyl ether-co-maleic anhydride), povidone, raffinose, shellac, sodium alginate, sodium starch glycolate, starch, pregelatinized starch, tragacanth and xanthan gum. For the purpose of delimiting the scope of the present invention without, however, restricting it, the term “rheological modifier” should be understood as a thickening agent or a viscosity modifying agent.

In an embodiment, the pseudoplastic thickening polymer (g) is selected from the group consisting of microcrystalline cellulose, starch, sodium starch glycolate, HPMC<MC, HPC, ethyl cellulose.

In an embodiment, the final viscosity of the composition is 10⁵ cP.

In an embodiment, the composition pH is between 5.0-6.0.

In an embodiment, the composition further comprises (h) at least one sweetener.

In an embodiment, the (h) sweetener is present in the composition in the range of 0.01-85 wt % of the composition.

In an embodiment, the sweetener is selected from the group consisting of acesulfame, aspartame, sodium cyclamate, saccharin, sucralose, neotame, thaumatin, neohesperidin, dextrose, sucrose, xylitol, maltitol, mannitol.

In a second object, the present invention shows the process for preparing a bio-adhesive nasal spray composition, as defined in the first object and in its embodiments, comprising the steps of:
(a) Preparation of the buffer system containing a thickening polymer;
(b) Addition of the active ingredient to the aqueous buffer system;
(c) Inclusion of a thermogelling agent and a preservative agent in the active ingredient;
(d) Preparation of micro/nanofibrillated cellulose;
(e) Mixing and homogenization of the systems described above; and
(f) Final volume adjustment via addition of purified water.

In an embodiment, the buffering system is selected from the group consisting of citric acid, sodium citrate, sodium phosphate monohydrate, sodium phosphate dihydrate, phosphoric acid, hydrochloric acid, sodium hydroxide, ammonium hydroxide, boric acid, and sodium borate.

In an embodiment, the buffering system is in a usage range of 0.01-5%, preferably 0.01-2%.

In an embodiment, the rheology modifying agents are selected from the group consisting of acacia, albumins, sodium carboxymethyl cellulose, carrageenans, microcrystalline cellulose, cellulose acetate, chitosan, dextrins, gelatin, guar gum, hyaluronic acid, hydroxyethyl cellulose, hydroxypropyl starch, hydroxypropyl cellulose, hydroxymethylpropyl cellulose, methyl cellulose, polyethylene glycols, poly(methyl vinyl ether-co-maleic anhydride), povidone, raffinose, shellac, sodium alginate, sodium starch glycolate, starch, pregelatinized starch, tragacanth, and xanthan gum.

In an embodiment, the rheological modifying agents are in a usage range of 0.01-40%, preferably 0.01-10%.

In an embodiment, the preservative agent is selected from the group consisting of cetrimide, cetylpyridinium chloride, chlorhexidine, chlorobutanol, chlorocresol, chloroxylenol, cresol, dimethyl ether, ethylparaben, glycerin, hexetidine, imidazolidinyl urea, methylparaben, phenoxyethanol, phenethyl alcohol, potassium benzoate, potassium metabisulfite, potassium sorbate, propionic acid, propylparaben, sodium benzoate, sodium metabisulfite, and thimerosal.

In an embodiment, the preservative is in a usage range of >20%, preferably 0.01-2%.

In an embodiment, the preparation of micro/nanofibrillated cellulose (d) is from homogenization under high shear provided by a rotor-stator of 6-7 cm diameter at a speed between 5,000-15,000 rpm, preferably at 8,000-10,000 rpm, temperature between 40-90° C., preferably initially employing 40-50° C. and keeping heating up to 85° C. for 10-20 minutes.

In an embodiment, the preparation of micro/nanofibrillated cellulose (d) is from homogenization under high shear provided by a rotor-stator at a speed of 10,000 rpm, initial temperatures of 40-50° C. and final temperatures of 85° C. for 20 minutes.

In an embodiment, the sweetener is selected from the group consisting of: acesulfame, aspartame, sodium cyclamate, saccharin, sucralose, neotame, thaumatin, neohesperidin, dextrose, sucrose, xylitol, maltitol, mannitol.

In an embodiment, the sweetener is in the usage range of 0.01-85%, preferably 0.01-10%.

In an embodiment, purified water is in the 20-99% usage range.

In a third object, the present invention shows the use of the bio-adhesive nasal spray composition, as defined in the first object and in its embodiments, for the manufacture of a medicament for the treatment of nausea and vomiting.

The differential of the present invention is the use of microcrystalline cellulose and internal processing to obtain a mixed system, containing a fraction of fibrillated cellulose in order to have a unique pseudoplastic behavior favorable in nasal formulations.

The microfibrillated celluloses of the present invention are produced by passing a liquid suspension of microcrystalline cellulose through a high-speed grinder (shearer) followed by a progressive deceleration thereof.

The present invention also shows a sweetening system aimed at masking the flavor of the composition of the present invention. It is expected that, with greater retention in the mucosa a very small amount of product can reach the larynx and oral cavity. Even so, excipients were added that will provide a more pleasant flavor, especially after the residence time in the nasal mucosa with mucociliary clearance. This characteristic is an additional factor in the greater adherence of patients to treatment with the product targeted by this document.

Regarding the desired properties of the preparation of the present invention, the following aspects are guaranteed: drug concentration between 1 to 15 mg/mL; pH within the physiological range (5.0-7.0), close to the isotonicity range, keeping the drug stable (physically and chemically) for a prolonged period (over 24 months); has non-ionic characteristics (favoring the stabilization of the drug), suspension with pseudoplastic behavior that allows for atomization with small droplets and rapid increase in viscosity in contact with the mucosa, in addition to thermogelling, with increase in viscosity at body temperature.

All of these aspects described were chosen to keep the product in contact with the mucosa for as long as possible and enhance absorption. In addition, one of the components of the sweetening system (xylitol) also acts as a tonicity agent, making the product slightly hypertonic.

The difference, considering the use of common materials, is the pseudoplastic behavior (the viscosity of the system decreases with increasing shear, that is, “shear thinning”) obtained in situ by combining the composition and processing conditions of the various excipients.

### EXAMPLES

The following examples are to illustrate aspects of the present invention without, however, having any limiting character.

### Example 1—Bio-Adhesive Nasal Spray Composition

A preferred, non-limiting embodiment of the composition of the present invention, together with all its manufacturing parameters, is presented in Table 1.

TABLE 1
Nasal spray composition of the present invention.

| Components | Quantities (mg/mL) | | |
|---|---|---|---|
| 5-15 mg/mL (base) | mg/mL | mg/mL | Function |
| Granisetron | 16.8 | 5.6 | Active |
| Hydrochl Inj | | | ingredient |
| Microcrystalline | 30 | 30 | Thickening |
| Cellulose | | | agent |
| Sodium Citrate | 11.91 | 11.91 | Buffering |
| 2H2O USP | | | agent |
| Anhydrous Citric Acid | 2 | 2 | Buffering |
| Fine Powder USP | | | agent |
| Methyl cellulose A4M | 3 | 3 | Thickening |
| | | | agent |
| Benzalkonium Chloride | 0.4 | 0.4 | Preservative |
| Solution 50% | | | |
| Xylitol 300 | 45 | 45 | Sweetener |
| White Glycerin USP | 50 | 50 | Thickening |
| | | | agent |
| Sodium Saccharin USP | 3 | 1.5 | Sweetener |
| Neohesperidin | 0.8 | 0.4 | Sweetener |
| Purified water q.s.p. Lt. | q.s.p. 100 mL | q.s.p. 100 mL | Vehicle |
| Density = 1.045 | Total | Total | |

Among the components of the mentioned composition, their functions in the proposed composition are described below.

Among cellulose derivatives, methylcellulose has relative hydrophobicity and the ability, under favorable ionic conditions, to have a thermogelling behavior with increasing temperature (25° C.→35° C.), even at low concentrations. Methylcellulose (MC) is a cellulose derivative, which is water-soluble and transforms into a gel at a particular temperature due to the hydrophobic intermolecular interaction. The MC gel is completely thermoreversible, being gelled with heating and liquefied with cooling of the composition. The final viscosity of the methylcellulose-based gel depends on the degree of substitution and the molecular weight of the active substance (molecule) used. In addition to its thermal reversibility, methylcellulose undergoes swelling and erosion in vivo, so it is not necessary to remove the gel after complete release of the drug from the applied area. Thus, MC is biocompatible and safe for application in drug delivery.

The choice of preservative was based on the pH of the composition (5.0-6.0), which excluded many materials, and compatibility with the drug and other components. It is known that large amounts of preservatives in nasal formulations can cause damage to the mucosa, such as nasal congestion and hypersensitivity. In order to avoid such damage, it was decided to use low doses of benzalkonium chloride (BZK), less than 0.2 wt % of the composition, a percentage that does not constitute damage to the ciliary mucosa. In addition, a pH of around 4.5 to 5.5 also favors the preservation of the nasal mucosa.

To optimize the viscosity of the system, it was decided to use materials that provide a more complex rheological behavior than a simple linear increase in this property. Pseudoplastic fluids have rheological properties similar to water during droplet generation (shear stage), but viscosity that can be controlled by shear stress before and after atomization.

The composition presented in Table 1 shows a differentiated behavior, since, by combining polymers and asymmetric cellulose particles, under specific preparation conditions, a system is obtained that allows the formation of drops (atomization) for the wide coverage of the nasal mucosa, while at the same time allowing for a rapid elastic recovery by drastically increasing its viscosity at rest. Another advantage, in detriment to co-processed excipients on the market that have polymeric mixtures, lies in the fact that the composition provides a simple approach to combine the advantages of different materials, for example, adhesiveness and temperature responsiveness. The approach of mixing polymeric additives is advantageous, since the resulting materials do not require the regulatory burden that a chemical modification would result in, nor the impact that a change of suppliers can cause, in the event of a supply disruption.

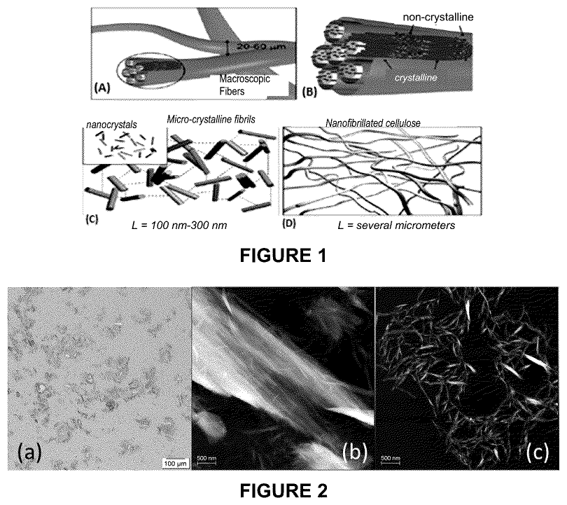

The in-situ production of micro/nanocellulose particles from microcrystalline cellulose was performed by exposing the outer layer of the original fibers by mechanical shearing, exposing the bundles of micrometric fibrils. These fibrils are much smaller in diameter compared to the original fibers, as shown in FIG. 1. The macroscopic fibers are mechanically sheared until the fibrils are released. These microfibrils, when in suspension, can form a network or web-like structure. The rheological properties obtained through the presence of microcrystalline cellulose particles are intrinsic to their structure, a fact that can be verified by comparing the properties of mechanically untreated and treated fibers. Untreated fibers have macroscopic dimensions and separate-by precipitation-from the water when kept stationary. On the other hand, when in an aqueous medium, the suspension containing micro/nanofibrillated cellulose forms a uniform gel of high viscosity. The fibrils are hydrophilic and the hydroxyls on their surface bind to water molecules, which allows this material to “retain” water, making it advantageous to use this material as a thickener due to its high surface area.

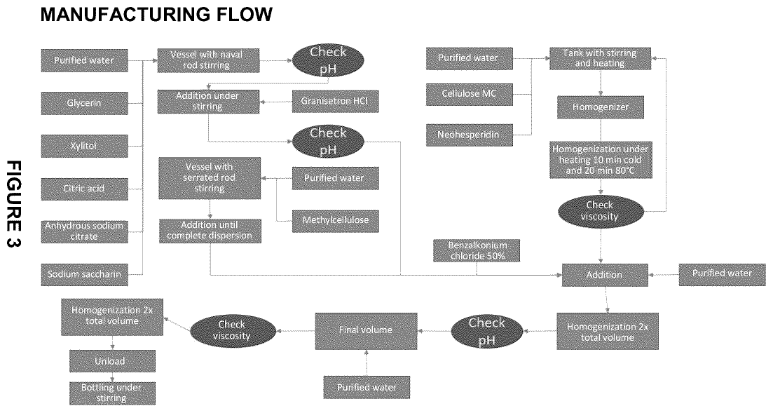

The success of nanofibrillation and the rheological properties of the gel described herein are closely linked. Optimized processing conditions used to obtain ideal fibrillation of the material led to the observation of micro and nanoparticle fractions (FIG. 2). The total cellulosic fraction in the suspension should contain between 0.5-2.0% nanoparticles, with fractions between 0.75-1.25% being preferable. These values can be obtained gravimetrically by analyzing the supernatant of the cellulosic suspension after centrifugation for 3 minutes at a G force of 1,000.

### Example 2—Process for Obtaining the Bio-Adhesive Nasal Spray Composition

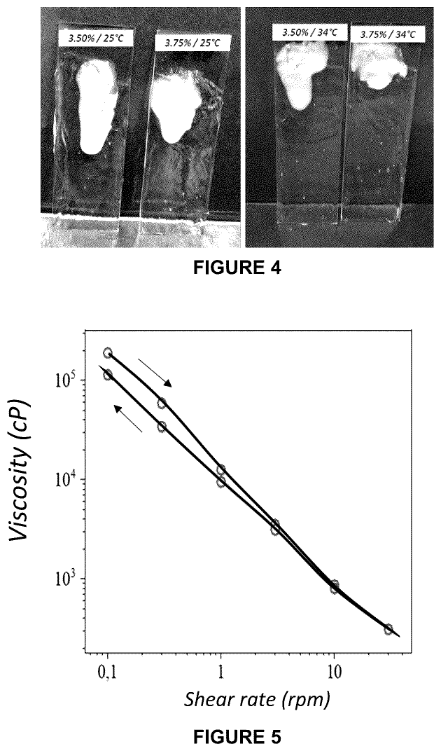

The composition obtained is the result of the combination of the properties of methyl cellulose with micro/nanofibrillated cellulose (MFC). This material is not commercially available for use in pharmaceutical products, but after adequate processing during the preparation of the composition in question (FIG. 3), it was possible to obtain an intermediate material capable of promoting the characteristic rheological behavior shown in FIG. 4.

In the process of obtaining the nasal spray composition of the present invention, the preparation of the buffering system consists of adding the material in a qsp quantity of purified water under stirring until complete dissolution. A mechanical stirrer with a naval rod is used at room temperature.

The addition of the active ingredient to the aqueous buffer system occurs under constant stirring until complete dissolution.

The preparation of the thermogelling system consists of adding the material in a qsp quantity of purified water under stirring until complete dispersion. A mechanical stirrer with a serrated rod is used at room temperature (below 30° C.).

In this process, microfibrillated celluloses are produced by passing a liquid suspension of microcrystalline cellulose through a high-speed grinder (shearer) followed by a progressive deceleration of the speed. The process is repeated until the suspended cellulose becomes a stable system and reaches a “gel point”. The process converts commercial cellulose into microfibrillated cellulose without substantially altering the chemical composition of the starting material. In this process, the material is not chemically degraded and its degree of polymerization remains substantially unchanged. On the other hand, the product obtained has a higher degree of fibrillation and greater accessibility of its surface than other known cellulosic products. If micro/nanofibrillation is successfully performed, the system will exhibit pseudoplastic behavior, with viscosities between 10⁴ and 10⁶ cP, preferably with viscosities close to 10⁵ cP, as exemplified in FIG. 5, for a suspension containing 3.5% microcrystalline cellulose.

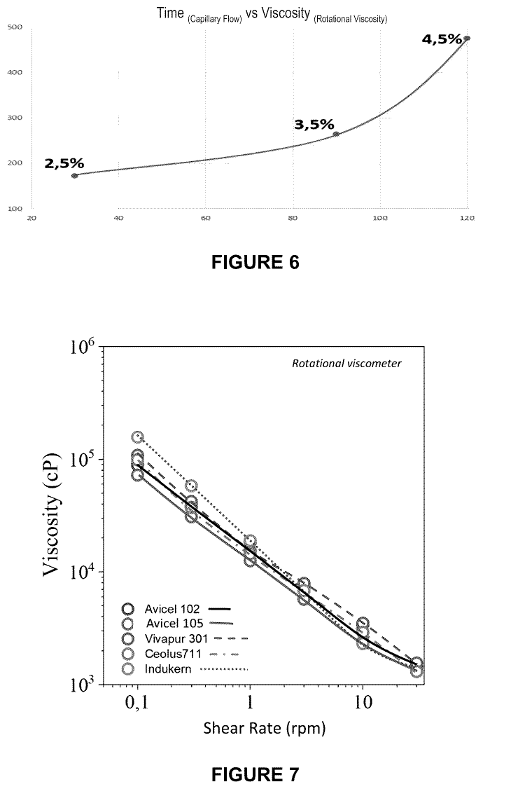

The material obtained has a direct correlation with the flow and final viscosity, with an exponential increase in the capillary flow time of the suspension in question from 3.0% w/w in solution (FIG. 6).

The processing consists of adding a 10% w/w microcrystalline cellulose suspension to a suitable container, starting homogenization using Ultra-turrax equipment (stator rotor must have an opening smaller than 0.25 mm) at 10,000 rpm at room temperature for 10 min. At the end of this step, the temperature will have increased to 40-50° C. At this point, the material has low viscosity, but the cellulose present will be widely dispersed, without lumps. While keeping stirring (grinding), the material is heated and reaches 85° C. for 20 min. At the end of the process, the material will acquire high viscosity and will have a “gel” characteristic. This material is added to the other components of the product and finely dispersed to obtain a stable product. The complete manufacturing process is described in FIG. 3 (Hiltunen, S. et al., 2019; Turbak et al., 1983).

The critical step of the process described above is, in fact, the dispersion of MC cellulose in water. This step is conducted in a separate container and its success is easily verified by the change in the appearance of the suspension, which changes from a fluid liquid to a high-viscosity gelled material. The process described was applied to different formulations, with the main focus on optimizing the concentration of materials such as microcrystalline cellulose used initially, its degree of dispersion and the amount of salts present. In general, the final viscosity of the solution is dominated by the presence of microcrystalline cellulose, as shown in FIG. 7.

A crucial point in obtaining a cellulosic system with micro/nanofibrillated characteristics is strongly related to the source of the cellulose used. This occurs for two main reasons: the first, already mentioned, is the relationship between the length and diameter of the microparticles used. It is the asymmetry of these particles that gives the desired pseudoplastic character in the suspension, being essential for the desired rheological behavior; the second, more delicate, is the ease with which the particles are deconstructed. This factor is strongly dependent on the physical characteristics of the original fibers. (Pinto et al., 2019.)

### Example 3—Physical-Chemical Tests

FIG. 4 shows the gelling potential of the material on a plate coated with a polymer that simulates the nasal mucosa. The behavior of the material in this static state is ideal for the effective release of the active ingredient, without the composition flowing. FIG. 4 also shows how the viscosity of the composition is modified according to the initial fraction of microcrystalline cellulose present in it and the temperature of the system, a consequence of the presence of methyl cellulose.

The measurement of the viscosities of these formulations can be seen in FIG. 5, which exemplifies a series of viscosimetric experiments demonstrating that the systems present initial viscosities of around 10⁵ cP, with a remarkable drop when shear increases. This demonstrates that all the formulations presented have a pseudoplastic behavior, which drop in viscosity allows the extraction of the liquid from inside the bottle, helping to form a spray with ideal characteristics. No hysteresis effects were observed, demonstrating that the liquid completely recovers its initial viscosity (at rest) after being forced through the spray valve. This behavior is directly reflected in how the product behaves when atomized from a metering valve. It is a premise that a product for nasal use can be applied broadly and directed over the nasal mucosa.

Additionally, the experiments demonstrate small variations in viscosity between temperatures of 25° C. and 34° C., as indicated in FIG. 4. It can be observed that at the temperature of the nasal cavity (approximately 34° C.), the suspension will present a slight increase in viscosity, reducing its flow.

A quantitative relationship between the viscosity of the system (measured by rotational viscometer) and its flow time (capillary viscometer) is also shown in FIG. 6. A direct relationship can be observed between the concentration of microcrystalline cellulose and the tendency of the material not to flow. This relationship suggests that an increase in viscosity at low speeds can simulate the flow of the fluid under the action of gravity and atmospheric pressure, as found in the nasal cavity of the patient. This result demonstrates the strong effect of the concentration of the micro/nanocellulose suspension on the desired properties of the final formulation.

FIG. 7 shows the behavior of the composition using different grades of microcrystalline cellulose processed to obtain a micro/nanofibrillated system. In all cases studied, a rheological behavior exceptionally different from that expected for a simple cellulose suspension (e.g., HPMC, MC, etc.) was observed. However, the viscosity values found varied widely according to the grade of cellulose used. More than that, the visual aspect of the suspensions and the time required to confer the desired viscosity to them were radically different.

Given the solid-state properties, particular to each of the microcrystalline celluloses presented, it was observed that materials with lower crystallinity, larger particle size and presenting a certain range of degree of polymerization offer better performance at lower concentrations. Thus, it was defined that the degree of crystallinity of the material should be less than 80%, preferably less than 78%; real density less than 1.62 g/cm³, preferably between 1.6 and 1.62 g/cm³; degree of polymerization between 210-250, preferably between 215-245. Additionally, the determination of the particle size distribution via light scattering indicates the use of microcrystalline celluloses smaller than 270 (d90), preferably between 230-270 microns. The resulting viscosity is above 150,000 cP for dispersion at 10% w/v.

These materials result in suspensions containing between 0.5 and 2.0% of micro/nanoparticles that present a fibrillar morphology characteristic of these materials (FIG. 2). Due to this asymmetric nature and small dimensions, these nanoparticles were characterized via atomic force microscopy. The measurement of approximately 500 particles indicates lengths between 100 and 2,000 nm, mostly between 200 and 500 nm.

Considering all the criteria defined to optimize the excipient platform, as described above, the final composition was characterized in terms of physical behavior (spraying and droplet distribution) and content and degradants after exposure to accelerated temperature conditions. The stability data of the composition exposed to different conditions are also an indication that the choice of excipients and their combination are favorable to obtaining a product of adequate pharmaceutical quality. All materials used do not have a charge in solution, which provides greater physical stability to the system and keeps the drug solubilized and stable throughout the primary packaging period. No sharp drop in content or formation of degradants was observed, even in the most critical condition at 50° C.

TABLE 2
Analysis data on granisetron HCl content and main impurities in samples exposed to accelerated stability
conditions (40° C. and 50° C.). All samples analyzed contained 5 mg/mL and one sample contained 15 mg/mL

| | Impurity | Impurity | Unknown | | Am 1 | Am 2 | Deviation | Average | |
|---|---|---|---|---|---|---|---|---|---|
| Degradation Product | C (%) | D (%) | Impurities (%) | Contents | (mg/mL) | (mg/mL) | (%) | (mg/mL) | % |
| GGG019/21 Control | NA | NA | NA | GGG019/21 Control | 5.25 | 5.24 | 0.15 | 5.25 | 105.00 |
| GGG019/21 40° C./30 d | 0.02* | NA | NA | GGG019/21 40° C./30 d | 5.21 | 5.2 | 0.19 | 5.21 | 104.20 |
| GGG019/21 50° C./30 d | 0.01* | NA | NA | GGG019/21 50° C./30 d | 5.21 | 5.22 | 0.11 | 5.22 | 104.40 |
| GGG026/21 (15 mg/mL) | 0.02* | NA | NA | GGG026/21 (15 mg/mL) | 14.97 | 15.02 | 0.24 | 14.99 | 99.97 |
| GGG027/21 | 0.02* | NA | NA | GGG027/21 | 5.07 | 5.07 | 0.01 | 5.07 | 101.40 |

*Detected, but below the 0.1% notification threshold.

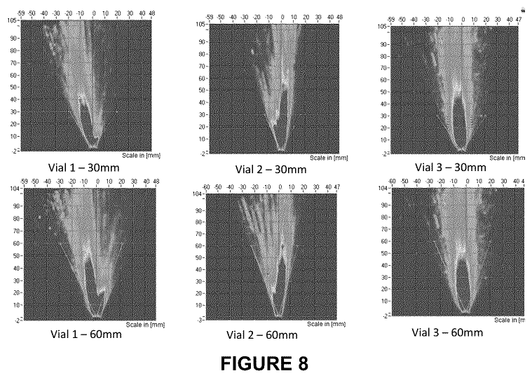

The data obtained through the characterization of the droplet size by atomization of the composition were also shown to be adequate, as demonstrated in Table 3 and FIG. 8. These values are obtained by analyzing the laser diffraction of the plume formed after atomization of the suspension through an applicator similar to that used in this type of products. The viability of the proposed composition is clearly observed in the average droplet size distribution (d50), with values between 110 and 130 microns as recommended by the EMA and FDA for nasal products.

TABLE 3
Droplet size distribution obtained from the composition presented above. A 100 μL vial
with a dosing valve was used. The data were obtained using a Spray Tec ® (Malwern) device

| Vial | | | | | Part. | Vial | | | | | Part. |
|---|---|---|---|---|---|---|---|---|---|---|---|
| (30 mm) | D10 | D50 | D90 | SPAN | <10 μm | (60 mm) | D10 | D50 | D90 | SPAN | <10 μm |
| Vial 1 | 44.89 | 137.60 | 280.40 | 1.71 | 0.00 | Vial 1 | 46.01 | 130.20 | 265.50 | 1.685 | 0.00 |
| Vial 1 | 62.37 | 152.10 | 315.50 | 1.66 | 0.00 | Vial 1 | 44.55 | 130.20 | 265.10 | 1.693 | 0.00 |
| Vial 2 | 44.71 | 130.00 | 273.70 | 1.76 | 0.00 | Vial 2 | 36.33 | 98.74 | 209.00 | 1.748 | 0.25 |
| Vial 2 | 55.56 | 145.10 | 290.60 | 1.62 | 0.00 | Vial 2 | 41.67 | 108.40 | 222.60 | 1.669 | 0.18 |
| Vial 3 | 36.40 | 111.20 | 230.10 | 1.74 | 0.02 | Vial 3 | 45.75 | 112.60 | 233.20 | 1.665 | 0.19 |
| Vial 3 | 32.22 | 102.00 | 221.40 | 1.86 | 0.06 | Vial 3 | 33.29 | 93.39 | 214.70 | 1.943 | 0.00 |
| Average | 46.03 | 129.67 | 268.62 | 1.73 | 0.01 | Average | 41.27 | 112.26 | 235.02 | 1.73 | 0.10 |
| DPR (%) | 24.66 | 15.08 | 13.48 | 4.74 | 181.66 | DPR (%) | 12.89 | 13.79 | 10.56 | 6.16 | 111.97 |

The proposed system is intended for the manufacture of pharmaceutical products for intranasal drug administration. It can be easily scaled up for industrial production with larger preparation volumes. The proposed system can be used for intranasal administration of any drug, with any ionic characteristic (anionic, cationic, or amphiphilic); The proposed system preserves viscosity characteristics at any pH between 4.0-7.5.

It should be understood that the embodiments described above are merely illustrative and that any modification along them may occur to a person skilled in the art. Consequently, the present invention should not be considered limited to the embodiments described herein.

1. A bio-adhesive nasal spray composition comprising:
(a) at least one active pharmaceutical ingredient;
(b) at least one tonicity agent;
(c) at least one preservative;
(d) at least one buffering system;
(e) at least one rheological modifier;
(f) at least one pseudoplastic thickening polymer; and
(g) micro/nanofibrillated cellulose.

2. The composition according to claim 1, wherein it comprises:
(a) at least one active pharmaceutical ingredient in a range of 0.01-5 wt % of the composition;
(b) at least one tonicity agent in a range of 0.1-20 wt % of the composition;
(c) at least one preservative in a range of 0.001-5 wt % of the composition;
(d) at least one buffering system in a range of 0.1-5 wt % of the composition;
(e) at least one rheological modifier in a range of 0.01-10 wt % of the composition;
(f) at least one pseudoplastic thickening polymer in a range of 0.01-25 wt % of the composition; and
(g) micro/nanofibrillated cellulose in a range of 3.0-4.0% by mass of the composition,
final viscosity of which ranges from 10⁴-10⁶ cP.

3. The composition according to claim 1, wherein the composition pH is between 5.0-6.0.

4. The composition according to claim 1, wherein it further comprises (h) at least one sweetener.

5. The composition according to claim 4, wherein characterized the sweetener (h) is in the range of 0.01-85 wt % of the composition.

6. A process for preparing a bio-adhesive nasal spray composition as defined in claim 1, wherein the process comprises the steps of:
(a) Preparation of the buffer system containing a thickening polymer;
(b) Addition of the active ingredient to the aqueous buffer system;
(c) Inclusion of a thermogelling agent and a preservative agent in the active ingredient;
(d) Preparation of micro/nanofibrillated cellulose;
(e) Mixing and homogenization of the systems described above; and
(f) Adjustment of final volume via addition of purified water.

7. The process for preparing a bio-adhesive nasal spray composition according to claim 6, wherein the preparation of the micro/nanofibrillated cellulose (d) is from homogenization under high shear provided by a rotor-stator of 6-7 cm diameter at a speed between 5,000-15,000 rpm, preferably at 8,000-10,000 rpm, temperature between 40-90° C., preferably initially employing 40-50° C. and keeping heating up to 85° C.

8. The process for preparing a bio-adhesive nasal spray composition as defined in claim 6, wherein the preparation of micro/nanofibrillated cellulose (d) is from homogenization under high shear provided by a rotor-stator at a speed of 10,000 rpm, initial temperatures of 40-50° C. and final temperatures of 85° C. for 10-20 minutes.

9. A method for a manufacture of a medicament for a treatment of nausea and vomiting using the bio-adhesive nasal spray composition of claim 1.
